SEQUENCE LISTING (5 sequences)

SEQ ID NO: 1 - DNA, 445 bp, E. coli
accgcgtcgacaagaaggagatatacatatggcgtcgttccagtcgttcggcatcccgggccagctggaagtcatcaagaaggcgctggatcacgtgcgcgtcggcgtggtcatcaccgatcccgcgctggaagataacccgatcgtctacgtgaaccagggcttcgtgcagatgaccggctacgagaccgaggaaatcctgggcaagaacgcgcgcttcctccaggggaagcacaccgatccggcggaagtggacaacatccgcaccgcgctgcaaaataaagaaccggtcaccgtgcagatccagaactacaagaaggacggcacgatgttctggaacgaactgaacatcgatccgatggaaatcgaggataagacgtatttcgtcggcatccagaacgacatcaccaagcagaaggaatatgaaaagctgtgactgcaggac

SEQ ID NO: 2 - DNA, 538 bp, E. coli
accgcgtcgacaagaaggagatatacatatgaaatacctgctgccgaccgctgctgctggtctgctgctcctcgctgcccagccggcgatggccatggatatcggaattaattcggatccgatggcgtcgttccagtcgttcggcatcccgggccagctggaagtcatcaagaaggcgctggatcacgtgcgcgtcggcgtggtcatcaccgatcccgcgctggaagataacccgatcgtctacgtgaaccagggcttcgtgcagatgaccggctacgagaccgaggaaatcctgggcaagaacgcgcgcttcctccaggggaagcacaccgatccggcggaagtggacaacatccgcaccgcgctgcaaaataaagaaccggtcaccgtgcagatccagaactacaagaaggacggcacgatgttctggaacgaactgaacatcgatccgatggaaatcgaggataagacgtatttcgtcggcatccagaacgacatcaccaagcagaaggaatatgaaaagctgtgactgcaggac

SEQ ID NO: 3 - DNA, 577 bp, E. coli
accgcgtcgacaagaaggagatatacatatgaacaataacgatctctttcaggcatcacgtcggcgttttctggcacaactcggcggcttaaccgtcgccgggatgctggggccgtcattgttaacgccgcgacgtgcgactgcggcgcaagcgggatccatggcgtcgttccagtcgttcggcatcccgggccagctggaagtcatcaagaaggcgctggatcacgtgcgcgtcggcgtggtcatcaccgatcccgcgctggaagataacccgatcgtctacgtgaaccagggcttcgtgcagatgaccggctacgagaccgaggaaatcctgggcaagaacgcgcgcttcctccaggggaagcacaccgatccggcggaagtggacaacatccgcaccgcgctgcaaaataaagaaccggtcaccgtgcagatccagaactacaagaaggacggcacgatgttctggaacgaactgaacatcgatccgatggaaatcgaggataagacgtatttcgtcggcatccagaacgacatcaccaagcagaaggaatatgaaaagctgtgactgcaggac

SEQ ID NO: 4 - DNA, 732 bp, E. coli
attggatccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagctcgagtac

SEQ ID NO: 5 - DNA, 808 bp, E. coli
tatgaaatacctgctgccgaccgctgctgctggtctgctgctcctcgctgcccagccggcgatggccatggatatcggaattaattcggatccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagc

(19) United States
(12) Patent Application Publication
(10) Pub. No.: US 2014/0227698 A1
(43) Pub. Date: Aug. 14, 2014

(54) USE OF FMN-BINDING FLUORESCENCE PROTEINS (FBFP) AS NEW TYPES OF SECRETION MARKERS

(71) Applicants: Thomas Drepper, Stolberg (DE); Karl-Erich Jaeger, Mülheim (DE); Janko Potzkei, Düsseldorf (DE)

(72) Inventors: Thomas Drepper, Stolberg (DE); Karl-Erich Jaeger, Mülheim (DE); Janko Potzkei, Düsseldorf (DE)

(73) Assignee: EVOCATAL GMBH, Monheim am Rhein (DE)

(21) Appl. No.: 14/237,282

(22) PCT Filed: Jul. 27, 2012
(86) PCT No.: PCT/EP2012/064776

§ 371 Date: Apr. 25, 2014

(30) Foreign Application Priority Data
Aug. 5, 2011 (DE) 10 2011 118 025.0

(51) Int. Cl. G01N 33/52; C12Q 1/68; C07K 14/245

(52) U.S. Cl. CPC G01N 33/52; C07K 14/245; C12Q 1/68
USPC 435/6.11; 530/350; 435/7.1

(57) ABSTRACT

To investigate protein-protein interactions, protein foldings and protein localization and also in the secretion of proteins, in vivo reporter proteins are used in biotechnology and in basic research. In order to be able to utilize fluorescence reporters as markers for secretion processes, FMN-binding fluorescence proteins (FbFP) have been developed by us for the first time. The new fluorescence markers can be expressed like GFP in various bacteria. The binding of the chromophore FMN produces a cyan-green fluorescent protein which can be detected in vivo using all customary spectroscopic and microscopic methods. In contrast to GFP, this protein can also surprisingly be secreted via the Sec route and be converted to the fluorescence-active state in the periplasma.

### REFERENCE TO RELATED APPLICATIONS

This application is a U.S. national stage application of International Patent Application No. PCT/EP2012/064776, filed Jul. 27, 2012, and claims the priority benefit of German Application No. 102011118025.0, filed Aug. 5, 2011, the entire disclosures of which are incorporated herein by reference.

### SEQUENCE LISTING

The instant application contains a Sequence Listing text file in ASCII format which is identical to the Sequence Listing text file submitted in International Patent Application No. PCT/EP2012/064776, on the international filing date of Jul. 27, 2012, and is hereby incorporated by reference in its entirety. The ASCII copy is entitled “MHTT6623.TXT”, was created on Aug. 5, 2011, and is 4739 bytes in size.

### FIELD OF THE INVENTION

To investigate protein-protein interactions, protein foldings and protein localization and also in the secretion of proteins, in vivo reporter proteins are used in biotechnology and in basic research. In order to be able to utilize fluorescence reporters as markers for secretion processes, FMN-binding fluorescence proteins (FbFP) have been developed by us for the first time. The new fluorescence markers can be expressed like GFP in various bacteria. The binding of the chromophore FMN produces a cyan-green fluorescent protein which can be detected in vivo using all customary spectroscopic and microscopic methods. In contrast to GFP, this protein can also surprisingly be secreted via the Sec route and be converted to the fluorescence-active state in the periplasma.

### BACKGROUND OF THE INVENTION

In vivo fluorescence markers such as GFP, are widely used in many areas of basic research and biotechnology: Fluorescent proteins are for instance being employed for investigating mechanisms of gene regulation or for monitoring biotechnological processes. Fluorescence markers can also be used to examine processes of cell differentiation and for localizing the respective target protein in the cell. They can also serve in examining folding processes of heterologous proteins in bacterial expression strains. In spite of the various application possibilities, the use of GFP as well as colour variants thereof (e.g. YFP (SEQ ID NO: 4) under anaerobic conditions is restricted, since oxygen is essential for the autocatalytic synthesis of the fluorophore. Thus GFP and its colour variants cannot be used in obligate anaerobic organisms. In order to nevertheless be able to employ fluorescence markers in vivo in anaerobic organisms, the FMN based fluorescent proteins (FbFP) have been developed. Without exception, all fluorescent proteins of the GFP family develop the chromophore in a biological multistage autocatalysis. As this process requires molecular oxygen, the maturation of the chromophore and thereby the formation of the fluorescence signal directly depends on this environmental factor. Consequently applications of GFP and its derivatives as fluorescence marker proteins are limited to aerobic systems, and the fluorescence signal of these proteins cannot be employed in obligate anaerobic organisms or under hypoxic conditions.

### BRIEF DESCRIPTION OF THE FIGURES

FIG. 1 shows a schematic representation of the fusions of the secretion reporter gene. Both a PelB signal sequence (2) and a TorA signal sequence (3) were fused to the reporter genes N-terminally. The reporter gene fusions are under control of the Lac-promotor.

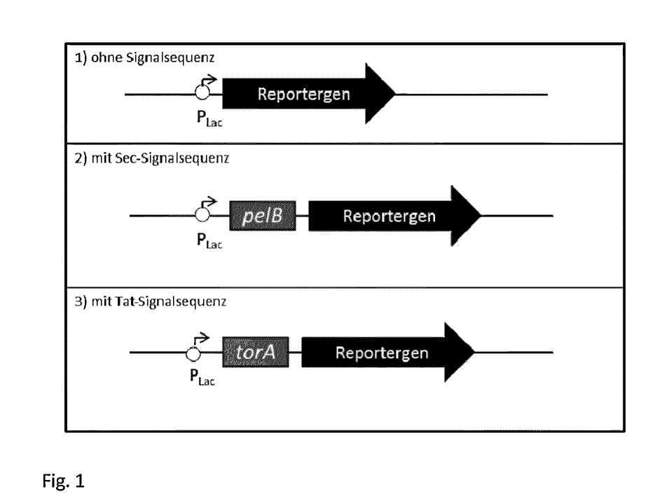

FIG. 2 depicts a cloning scheme for the formation of Sec-, and Tat-secretion fusions using the example of EcFbFP.

FIG. 3 depicts a localisation analysis by means of a fluorescence microscope; and

FIG. 4 depicts a further localisation analysis by means of a fluorescence microscope.

### SUMMARY OF THE INVENTION

It is therefore an object of the present invention to provide means for fluorescent labelling in the periplasm. Furthermore it is an object of the present invention to provide a fluorescence marker which can be transferred outwardly from the cytoplasm of a host organism and translocated, respectively, in an active form, i.e. in excitable form.

This object is solved by the use according to claim 1, as well as the method according to claim 14.

Accordingly, the use of a fluorescent protein as a secretion marker is suggested, wherein the fluorescent protein includes a LOV domain, in which at least one cycteine is replaced by another amino acid, which does not covalently bind to FMN.

### DETAILED DESCRIPTION OF THE INVENTION

The term “fluorescent protein” is understood to refer to a protein that is capable of emitting fluorescence. The fluorescence property can be caused by binding of a chromophore and a fluorophore, respectively, to certain regions of the protein, such as a LOV domain, or the fluorescence property is encoded in the peptide sequence of the protein, as for example with GFP. Fluorescence is being emitted after the fluorescent protein has been excited with light of certain wavelength. Mostly the excitation causes a brief, spontaneous emission of light upon transition of an electronically excited system to a state of lower energy, wherein the light emitted is generally of lower energy than the light previously absorbed.

The gene ytvA originating from Bacillus subtilis was identified within the scope of the complete genome sequencing, and classified as an unknown protein with similarity to protein kinases. In a study performed, on the basis of spectroscopic analysis it was assumed that YtvA might contain a chromophore in the form of a flavin mononucleotide (FMN). This assumption was confirmed in 2002, and by way of a data base search for proteins with homologies to plant phototropines, the N-terminal region of YtvA could be identified as a so called LOV domain (Light, Oxygen, Voltage). Phototropines are membrane-bound kinases of higher plants that autophosphorylate upon irradiation with blue (390-500 nm) and UV light (320-390 nm). In plants photoreceptors are responsible for a plant's phototropism, as well as the change of location of chloroplasts and the opening of stomata. Proteins with a LOV domain are generally regulated by the factors light, oxygen and voltage (Light, Oxygen, Voltage), where in bacteria they can be coupled to different effector domains. Furthermore LOV domains undergo a light-induced photo cycle. In contrast to phototropines, YtvA of a size of 261 amino acids consists of only two domains: an N-terminal LOV- and a C-terminal STAS-domain. For the YtvA LOV domain, which possesses the consensus sequence NCRFLQG, it could be shown that it binds to a FMN as a chromophore and undergoes a photo cycle, as LOV domains of the phototropines. It is assumed that the STAS domain of YtvA could be an effector-domain, which is responsible for forwarding the light stimulus sensed by the LOV domain.

The expression “Fluorescent protein comprising a LOV domain” shall in the following be understood as referring to a protein that includes a Light-Oxygen-Voltage (LOV) domain, in which at least one cysteine is replaced by another amino acid, and in which, in addition to the substitution of at least one cysteine, at least one further point mutation is present.

In contrast to GFP-like fluorescent proteins, LOV domain proteins bind to the cofactors FMN, FAD or riboflavin, which are being provided by the host organism. These molecules are being synthesised in an oxygen independent manner both in prokaryotes and eukaryotes.

On the basis of bacterial photoreceptors of the LOV family we have then developed a new family of FMN-binding fluorescence marker proteins (FbFP). FbFPs are recombinant variants of bacterial blue light receptors of the LOV (Light-Oxygen-Voltage) family. In contrast to the GFP-like fluorescent proteins the new fluorescence markers are very small (16-19 kDa) and bind to the chromophore Flavin-mononucleotide (FMN), provided by the host organism. This molecule is synthesised both in pro- and eukaryotes in an oxygen independent way. To increase FMN-dependent fluorescence of the blue light receptors and thereby allow their use as fluorescence markers, the bacterial proteins were altered by means of modern methods, the so called directed evolution. By way of these mutations, the autofluorescence of the proteins was drastically increased, resulting in the creation of the FMN binding fluorescent proteins. The photo-chemical characterisation of the new marker proteins showed that the FbFPs emit a bluish green fluorescence (495 nm) following excitation with blue light (450 nm). The new marker proteins could be expressed in different pro- and eukaryotic host cells, and the fluorescence characteristic for FbFP could be detected in vivo.

In form of the “FMN-based fluorescent proteins” (FbFPs) there are thus now fluorescence marker proteins, which fluoresce independent of oxygen partial pressure. In contrast to representatives of the GFP family, FbFP does not require oxygen for fluorescence generation. The expression “capable of fluorescence” shall in the following be understood to mean that a fluorescent protein can be excited by light of a certain wavelength and/or that it can release the energy absorbed upon excitation again.

About 20% of the polypeptides synthesised by bacteria are being transported out of the cytoplasm entirely or partially. Many of these proteins are hydrolases, such as e.g. lipases, proteases or carbohydrases, which serve in degrading natural substrates. This mainly serves in adapting the bacterial cell to altered conditions of the environment. However, toxins or components of so-called “quorum sensing systems”, which serve in sensing population density, are also being translocated out of bacterial cells. In this connection export and secretion of proteins are being distinguished: proteins to be exported remain, at least partially, within the external membrane, whereas a release into the extra-cellular medium occurs in secretion. In the following a brief overview shall be provided on the main mechanisms of bacterial secretion currently known. The type I- or ABC-secretion pathway (ATP-Binding Cassette) has been described for the first time for secretion of haemolysin in Escherichia coli. This transport system consists of three components: at the inner membrane there is the ATP binding cassette, which—besides its ATPase function—is also responsible for substrate specificity. In the periplasmatic space there is the Membrane Fusion Protein (MFP), which is fixed to the cytoplasm membrane via a large hydrophobic domain, and which interacts with the third component, the Outer Membrane Protein (OMP), which is located within the outer membrane. It is assumed that these three components together form an adhesion site between inner and outer membrane whereby a pore is being formed. The ABC substrates, which differ from most other secreted proteins by a non-cleavable C-terminal signal peptide, are being transferred through the periplasm into the extracellular medium in one step. In the general secretion pathway (General Secretory Pathway, GSP) of gram negative bacteria, via which most secretory proteins get outside, the protein to be secreted is being transferred from the cytoplasm to the ambient medium in two steps: in the first step the preprotein is being translocated into the periplasm via the Sec-pathway, upon cleavage of the N-terminal signal sequence. From there the protein can be translocated across the outer membrane. The protein binds to the “Main Terminal Branch” of the general secretion pathway, which consists of up to 14 different proteins. Another mechanism of reaching the ambient medium from the periplasm within the framework of the GSP, is used by the so called autotransporters: following their translocation into the periplasm via the Sec-secretion pathway the C-terminally situated β domain of the protein forms a channel in the outer membrane, consisting of several amphipathic β sheets, through which the N-terminal β domain of the protein can access the ambience. Since gram positive organisms, contrary to gram negative organisms, are endued with only a cytoplasm membrane, most proteins translocated via the Sec-pathway directly enter the ambient medium. In addition to the Sec system in both gram negative and gram positive organisms there exists the so called Tat-secretion pathway, which can—just as the Sec-pathway—transport proteins across the cytoplasm membrane. The largest portion of proteins secreted or exported by bacteria exits the cytoplasm via the Sec-translocation pathway. In the gram positive model organism Bacillus subtilis up to 300 different proteins can be transported out of the cytoplasm; in the process only a small portion uses routes of translocation such as the ABC, Pseudopilin- or Tat-translocation system, the largest part of the exported proteins is being secreted via the Sec-pathway. Most of the translocated proteins possess an N-terminal recognition signal, the so called signal sequence, the function of which is directing the proteins from the site of their synthesis to the cytoplasm membrane. The typical signal sequence of a Sec substrate consists of three different domains. At the N-terminus there is the so-called N domain. It is being assumed that the positive charges (arginine or lysine) interact with negative charges of the membrane pospholipides. Adjoining thereto there is the hydrophobic H domain. It consists of hydrophobic amino acids and can take an α-helical structure. In 60% of cases there is a helix breaking amino acid (proline or glycine) in the central region of the H domain; this allows the H domain to insert completely into the membrane according to the so-called “hairpin mechanism”. At the end of this domain mostly proline or glycine are found, which presumably improves cleaving off by signal peptidases. An important characteristic of the Sec-pathway is the translocation of unfolded proteins, with the unfolded state being essential for the translocation competence of a protein. With the help of cytoplasmic chaperones the synthesised polypeptides are being stabilised in an unfolded conformation, and are thus suitable for translocation. Folding of the proteins occurs only after the translocation process. In B. subtilis extracellular folding catalysts take high importance, because a number of proteases are located at the membrane/cell wall interface, so that proteins that are not folded or not correctly folded are degraded quickly. An example for this is the extracytoplasmatically located Lipoprotein PrsA, which shows sequence similarity to a peptidyl-prolyl-cis/trans isomerase of E. coli and could thus be regarded as a chaperone. As some proteins depend on the formation of disulfide bonds for their correct conformation, there is a need for thiol-disulfide oxidoreductases in the extra-cellular space. To this effect B. subtilis has at its disposal the proteins BdbA, BdbB and BdbC, wherein up to now only one extracytoplasmatic protein that contains a disulfide bridge is known, ComC. In E. coli disulfide bonds are formed in the periplasmatic space with the help of the Dsb apparatus, consisting of DsbA, DsbB, DsbC, DsbD, DsbE and DsbG. The largest portion of proteins exported from the cytoplasm in E. coli uses the Sec-pathway for translocation. To be translocation competent, the substrates of the Sec-pathway need to maintain an unfolded conformation after their synthesis. This poses a problem for many extracytoplasmatic proteins, which require a cofactor for their function, because binding of a cofactors is often accompanied by cytoplasmatic folding of the protein. A special translocation pathway for proteins with redoxactive cofactors in procaryotic organisms has been postulated already in 1996, since many periplasmatic enzymes, which included a cofactor such as molybdenum or FeS complexes, showed the Consensus motive S/T-R-R-X-F-L-K in their signal sequences. Such a secretion pathway was found in the thylakoid membranes of chloroplasts in the form of the pH dependent translocation pathway. Shortly afterward it was shown in E. coli that the periplasmatic triethylamine-N-oxidoreductase TorA, a molybdenum-containing enzyme, could be translocated from cyto- to periplasm in a manner independent of the Sec system. Due to the two arginines in the signal sequence of the substrates secreted in this way, this pathway was termed “Twin-arginine translocation” or Tat-secretion pathway. As it turned out, this Tat-pathway showed a high similarity to the pH dependent pathway of translocation found in thylakoid membranes, and is above all characterized by the ability to translocate proteins that are already folded. Similar to the translocation pathway in thylakoid membranes, it turned out for the bacterial Tat-pathway that the energy required for translocation solely originates from the PMF, which results from the proton gradient existing across the cytoplasm membrane. Together with the transport of folded proteins and the different signal peptides this is a major difference to the Sec-pathway. As also with the Sec system, the substrates of the Tat-pathway are being directed to the translocon by means of a signal peptide at the N-terminus of the protein to be secreted. The structure of the signal peptide corresponds to the one of a Sec signal sequence. The Tat signal peptide is classified into an N-, a H-, and a C-domain, and with 26 to 58 amino acids it is distinctly longer than the average Sec signal sequence, with a large portion of the additional amino acids being allotted to the N-domain. The N-domain contains the amino acid sequence S-R-R-X-F-L-K, with both arginines being highly conserved. Up to now only two natural Tat substrates are known that deviate from this motive. The remaining amino acids of the Consensus motive occur with a frequency of more than 50%, with X normally being a polar amino acid. It should furthermore be noted that the Tat signal sequence alone need not be sufficient for transferring a protein out of the cytoplasm in a Tat dependent way. An important factor is the folding state of the protein located at the signal sequence, since unfolded or misfolded proteins, or proteins with missing cofactor, are not being exported. The protein to be secreted in a Tat-dependent way should thus if possible not interact with chaperones of the Sec system and must be capable of being folded cytoplasmatically. Compared to the Sec-pathway, less is so far known about the translocon formed by the identified Tat components. It is postulated that in the membrane TatA, B and C exist in two different complex forms (TatAB and TatBC), which in case of a translocation combine to the complete translocon, consisting of TatA, B and C, wherein this complex represents only a transient state. In the process, the TatBC complex is supposed to take the role of the signal peptide receptor, whereas TatA is supposed to form the water filled pore necessary for the translocation, after binding of the signal peptide to TatBC. In case of a failure of the Tat system Tat substrates accumulate in the membrane and could thus lead to the observed phenotype of chain formation, which occurs in Tat mutants.

As already mentioned, in vivo fluorescence markers find widespread use in basic research as well as in biotechnology. However, representatives of the GFP family have a crucial disadvantage with regard to secretion studies: It could be shown that the fluorescent protein GFP can be transferred into the periplasm of E. coli in an active form by means of the Tat secretion pathway. However, it has so far not been possible to secrete the common representatives of the GFP family actively via the Sec-pathway, such that they cause a marked fluorescence in the periplasm. This is on one hand due to the reducing conditions that prevail in the periplasm, on the other hand upon folding cysteine residues form disulphide bonds, so that no correct folding of the FPs can occur in the periplasm. As a result the protein can be detected in the periplasm by means of Western Blot analysis, however, it is not present in a native and therefore fluorescence-active conformation.

In contrast to GFP, the fluorophore group of which is formed via an autocatalytic process, FbFPs require the cofactor FMN for their fluorescence. Since this cofactor is not covalently bound and is no cysteine is present in the protein, this makes the FbFPs potential Sec secretion markers and Tat secretion markers, respectively, for bacteria. As a protein, which for its function requires a cofactor that enters the periplasm from the media and which needs to be correctly folded, the FbFPs are optimal proteins for the Sec- and the Tat-pathway. To test the possibility of Sec- and Tat-dependent secretion of the FbFPs in E. coli, a Sec- and a Tat-signal sequence were fused to the EcFbFP (SEQ ID NO: 1). The PelB signal sequence served as a signal sequence of the Sec-pathway, the signal sequence of TorA was used for Tat dependent secretion.

A secretion marker for the purposes of the present invention is in particular a marker that can be used for detecting secretion of a protein, an enzyme and/or an antibody from a host organism. In the process, the detection may concern the general presence of secretion and/or its efficiency.

According to an embodiment of the invention at least one cysteine within the LOV domain is exchanged for alanine.

Furthermore it is preferred that the LOV domain includes at least one further point mutation in addition to the exchange of the at least one cysteine. Preferably the introduction of such a point mutation causes an improvement of the photo stability and/or a change of the fluorescence wavelength. This allows differential analysis and monitoring of the exported secretion markers capable of fluorescence.

Examples of suitable point mutations are, for instance, a point mutation from the group consisting of I29V, S91G, Y112F, E138G, L7P, F124L, N26Y, Y112H, I48T, H61Y, Y43F, Y112C, E12D, Q143L, A36T, Q57H, N95I, E22K, E71G, K88S, L109V and Q116L.

According to a further embodiment of the invention the fluorescent protein with a LOV domain is
(a) encoded by a nucleic acid of SEQ ID NO: 1 or a fragment, a variant, a homolog or a derivative of this sequence,
(b) encoded by a nucleic acid that can hybridise to one of the nucleic acids according to (a) under stringent conditions,
(c) encoded by a nucleic acid that includes at least 70%, preferably 95%, identity to one of the nucleic acids according to (a) or (b),
(d) encoded by a nucleic acid, which is capable of hybridizing to a nucleic acid that is complementary to one of the nucleic acids according to (a)-(c) under stringent conditions,
(e) encoded by a nucleic acid which permits at least one silent mutation of a single nucleotide (as permitted by the degeneracy of the genetic code), when compared to the nucleic acids according to (a)-(d), or
(f) it is encoded by a nucleic acid, the code of which was optimized for a certain expression system when compared the nucleic acids according to (a)-(e).

The term “nucleic acid” shall in the following be understood as referring to a single- or double-stranded macromolecule built up of nucleotides. The most common nucleic acids are desoxyribonucleic acid (DNA) and complementary DNA (cDNA), respectively, as well as ribonucleic acid (RNA). In DNA there are present the nucleic bases adenine, cytosine, guanine and thymine, the latter being specific for DNA. In RNA the same nucleic bases and nucleotides, respectively, are present, except for thymine, which is being replaced by uracil. Examples of artificial nucleic acids include peptide nucleic acid (PNA), morpholino and locked nucleic acid (LNA), as well as glycol nucleic acid (GNA) and threose nucleic acid (TNA). The build-up of the backbone of each of these nucleic acids differs from that of naturally occurring nucleic acids.

The term “complementary” shall be understood as referring to the nucleic acid complementary to the nucleic acid used/discussed. This is an important concept in molecular biology, because it concerns an important property of double-stranded nucleic acids such as DNA, RNA or DNA:RNA duplexes. One strand is complementary to the other, since the base pairs of the two strands are bound non-covalently by two or three hydrogen bonds. In principle—exceptions exist for thymine/uracil and the wobble complex of tRNA—there is only one complementary base for every base of a nucleic acid. Hence, it is possible to reconstruct the complementary strand of a particular strand. This is essential for example in DNA replication. As an example, the complementary strand of the DNA sequence would be

5′ A G T C A T G 3′
3′ T C A G T A C 5′.

In the case of DNA, the term “complementary” may also refer to cDNA. cDNA is synthesised from RNA, e.g., mRNA, by means of the enzyme reverse transcriptase.

The terms “hybridize” and “hybridization”, respectively, are in the following understood to refer to the process during which a more or less entirely complementary nucleic acid is being attached to a nucleic acid, by formation of hydrogen bonds between the respective complementary nucleic bases.

The term “hybridize under stringent conditions” is in the following understood to mean that the conditions of the hybridization reaction are adjusted in a way that only bases completely complementary to each other can form hydrogen bonds. Stringency can be controlled, for example, by the temperature.

The term “silent mutation” shall in the following be understood as referring to the phenomenon that a mutation in a nucleic acid segment has no consequences. This is the case as the information content of the gene has not changed, because a succession of amino acids can be encoded by amino acids by different groups of three consecutive nucleic bases—called triplets or codons.

The term “fragment” shall in the following characterize a part of a nucleic acid or an amino acid sequence, which lacks some portions of a claimed nucleic acid and amino acid sequence, respectively, which maintains, however, at least a part of its activity, e.g., with regard to fluorescence properties, enzyme activity or binding to other molecules.

The term “variant” shall in the following characterize a nucleic acid or an amino acid sequence, which in terms of structure and biological activity essentially resembles the structure and biological activity of a claimed nucleic acid or an amino acid sequence.

The term “derivative” is in the following understood to mean a related nucleic acid or amino acid sequence, which has similar characteristics as a claimed nucleic acid or amino acid sequence with regard to a target molecule.

The term “homolog” is in the following understood to mean a nucleic acid or amino acid sequence, in the sequence of which at least one nucleotide and one amino acid, respectively, is added, deleted, substituted or modified in another manner, when compared to the sequence of a claimed nucleic acid or amino acid sequence. A precondition is, however, that the homolog has essentially the same properties as a claimed nucleic acid or amino acid sequence.

The term “optimized for a particular expression system” shall in the following be understood as meaning that a nucleic acid is adapted to the codon usage of the organism in which it is to be expressed. The codon usage, also called the codon bias, refers to the phenomenon that different species often use variations of the universal genetic code at different frequency.

The term “sequence identity of at least X %” is in the following understood to mean a sequence identity that has been determined by a sequence comparison (alignment) by means of a BLAST algorithm, as available on the homepage of the NCBI.

According to a further embodiment of the invention the fluorescent protein has a size between ≧16 kDa and ≦19 kDa.

In a preferred embodiment of the invention the fluorescent protein has an excitation wavelength between ≧430 nm and ≦470 nm, preferably 450 nm. In this context it is preferred that the fluorescent protein has an emission maximum between ≧470 nm and ≦520 nm, preferably 495 nm.

According to a further embodiment of the invention the fluorescent protein is being expressed or co-expressed in a host cell, and being secreted into the periplasm and/or into an extracellular media. In this context co-expression within the meaning of the invention includes the expression as a fusion protein, as well as parallel expression together with a further protein. In this context it is furthermore preferably intended that the fluorescent protein includes a signal sequence at its N-terminus. As a signal sequence the fluorescent protein suitable for use as a secretion marker may for instance include a PelB- or a TorA-signal sequence.

It is in particular preferred that the fluorescent protein with a LOV domain
(a) is encoded by a nucleic acid of SEQ ID NO: 2 or 3, or a fragment, a variant, a homolog, or a derivative of one of these sequences,
(b) is encoded by a nucleic acid capable of hybridizing to the nucleic acid of (a) under stringent conditions,
(c) is encoded by a nucleic acid of at least 70% identity, preferably 95% identity, to a nucleic acid of (a) or (b),
(d) is encoded by a nucleic acid capable of hybridizing to a nucleic acid complementary to one of the nucleic acids of (a)-(c) under stringent conditions,
(e) is encoded by a nucleic acid that includes at least one silent mutation of a single nucleotide (as permitted by the degeneracy of the genetic code) when compared to the nucleic acids of (a)-(d), or
(f) is encoded by a nucleic acid, the code of which is optimized for a particular expression system when compared to the nucleic acids of (a)-(e).

In a preferred embodiment of the use an antibody expressed in an organism is labelled with the fluorescent protein that includes a LOV domain, in which at least one cysteine is replaced by another amino acid, preferably alanine, that does not covalently bind to FMN. This allows examining whether by the use of folding aides such as chaperones, an export of an antibody into e.g. the periplasm occurs in a desired manner. To this end it may be intended to fuse the fluorescent protein to the target protein, in order to detect the translocation of the target protein (antibody) across the membrane by means of fluorescent microscopic or spectroscopic methods. This is of relevance for detecting for instance antibodies or antibody fragments, which need to be secreted into the oxidizing periplasm in gram negative bacteria (such as e.g. E. coli), in order to form the disulphide bonds that are required there. A further application is detection in the context of high throughput screening methods.

Furthermore with the invention there is suggested a method for producing a secretion marker, wherein a plasmid that includes a ribonucleic acid encoding for a fluorescent protein is introduced into an organism, preferably a bacterium selected from the group consisting of Escherichia coli, Rhodobacter capsulatus, Pseudomonas putida and Bacillus subtilis, by means of methods of genetic engineering, and expressed there, wherein the fluorescent protein
(a) is encoded by a nucleic acid of SEQ ID NO: 1, 2 or 3, or a fragment, a variant, a homolog, or a derivative of one of these sequences,
(b) is encoded by a nucleic acid capable of hybridizing to the nucleic acid of (a) under stringent conditions,
(c) is encoded by a nucleic acid of at least 70% identity, preferably 95% identity, to a nucleic acid of (a) or (b),
(d) is encoded by a nucleic acid capable of hybridizing to a nucleic acid complementary to one of the nucleic acids of (a)-(c) under stringent conditions,
(e) is encoded by a nucleic acid that includes at least one silent mutation of a single nucleotide (as permitted by the degeneracy of the genetic code) when compared to the nucleic acids of (a)-(d), or
(f) is encoded by a nucleic acid, the code of which is optimized for a particular expression system when compared to the nucleic acids of (a)-(e).

It is in this context intended in a preferred embodiment of the method that as an expression vector at least one vector is used, which is selected from the group consisting of pRhotHi-2 and pHSG575.

### EXAMPLES

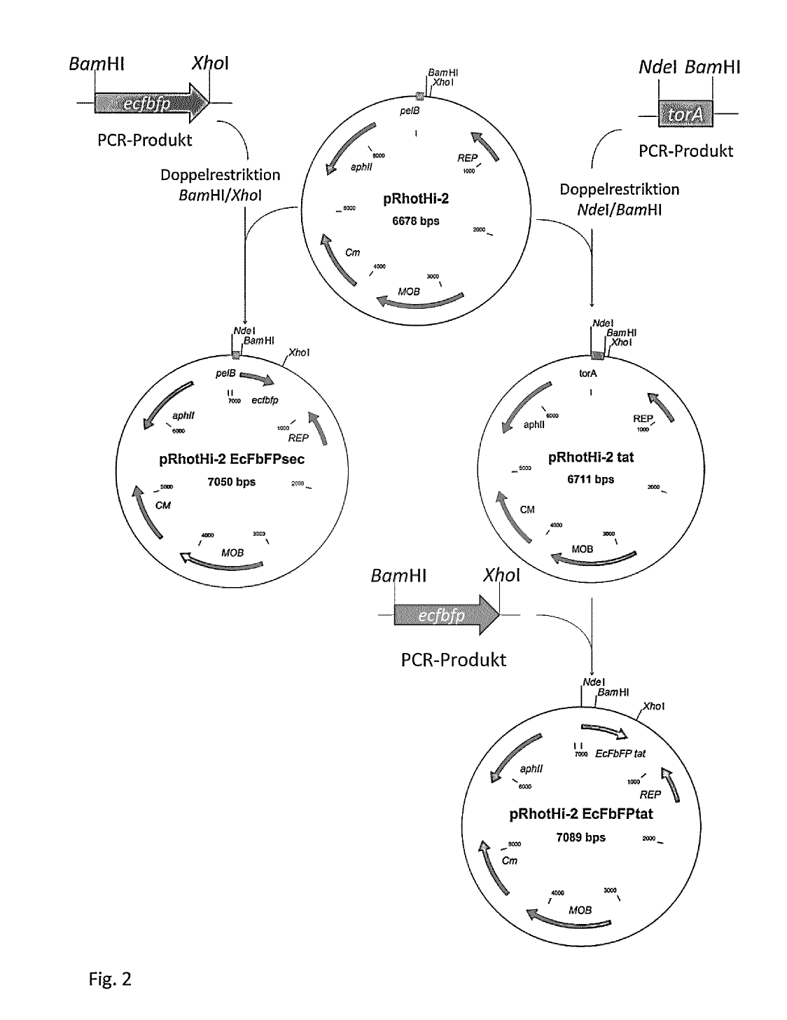

To test the secretion ability of the fluorescence marker protein in comparison to YFP (SEQ ID NO: 4), these proteins should first be cloned into the expression vector pRhotHi-2 and subsequently into the expression vector pHSG575. Due to the origin of replication (rep region, “broad host range origin of replication”) of pBBR1MCS the expression vector pRhotHi-2 possesses a wide host spectrum, and can be used in, e.g., R. capsulatus. For selection purposes the pBBR1MCS derivative contains a chloramphenicol resistance gene and a kanamycin resistance gene (aphII). For a potential use of the fusions in R. capsulatus a mob region allows plasmid transfer by means of conjugation via the E. coli strain S17-1 which acts as a donor strain into the R. capsulatus strain B10S and B10S-T7. For expression of the marker proteins it uses T7 Polymerase dependent promotor. The selected signal sequences of the Sec- and Tat-secretion pathway pelB and torA and the respective marker proteins were cloned into the expression vector pRhotHi-2, downstream of the T7 promotor (cloning strategy, see FIG. 2). In case of pelB only the respective marker proteins had to be cloned after the Sec signal sequence by means of restriction hydrolysis, because it was already included in the vector pRohtHi-2.

For synthesis of the expression plasmid with the Tat secretion sequence, torA was amplified using specific oligonucleotide starter molecules (“primers”) by means of PCR and equipped with a NdeI cleavage site at the 5′ end, as well as a BamHI cleavage site at the 3′ end, with the sequence of the NdeI cleavage site in addition encoding the start codon AUG. In this case, the genomic DNA of the E. coli strain k12 served as a template for the PCR reaction. The fluorescence marker gene YFP (SEQ ID NO: 4) was also amplified by means of PCR, and in addition the cleavage sites BamHI and XhoI were also inserted by means of specific primers. In both cases the stop codon was eliminated, since in the plasmid pRhotHi-2 downstream of the fusions there is a sequence encoding a His tag, by means of which the expressed proteins could possibly be purified in subsequent experiments. By way of a BamHI/XhoI dual restriction digest the Sec- and Tat-fusions, respectively, were cloned into the hydrolysed vector pRhotHi-2, and subsequently transformed into the bacterial strain E. coli DH5 α.

The positive result of the cloning was confirmed by means of a restriction analysis and was verified by sequencing. The respective constructs without signal sequence were used as an expression control.

FIG. 2 depicts a cloning scheme for the generation of Sec-, and Tat-secretion fusions using the example of EcFbFP (SEQ ID NO: 1). The example illustrates the cloning strategy by means of EcFbFP (SEQ ID NO: 1), the same was carried out using YFP (SEQ ID NO: 4). In case of the Sec fusion the PCR product of the marker protein was cloned by means of BamHI/XhoI double restriction into the hydrolysed vector pRhotHi-2. In addition to cloning the Tat fusion, which was cloned analogously to the Sec fusion, the PCR product of the Tat-secretion signal sequence torA was cloned by means of NdeI/BamHI double restriction into the hydrolysed vector in advance.

Expression of the constructs was initially performed under the control of the inducible Lac promotor in the vector pHSG575, which exists in low copy numbers in E. coli. This approach was to ensure that the secretion of the respective marker protein was not affected by a too excessive overexpression, which would lead to the formation of inclusion bodies. In addition, the vector carries the gene for resistance to chloramphenicol, in order to maintain selection pressure. EcFbFP (SEQ ID NO: 1), to the 5′-end of which no signal sequence was appended, served as an expression control.

To this effect, the secretion marker constructs were cloned into the vector pHSG575. To synthesize the expression plasmid with the respective secretion marker constructs, the EcFbFP (SEQ ID NO: 1) gene, the EcFbFPsec SEQ ID NO: 2) gene, and the EcFbFPtat (SEQ ID NO: 3) gene were amplified from the respective pRhotHi-2 construct by means of PCR, and equipped with a SalI cleavage site at the 5′ end and a PstI cleavage site at the 3′ end by means of specific primers. The PCR products, hydrolysed with the respective restriction endonucleases, were cloned into the likewise hydrolysed vector pHSG575, and the successful cloning was verified by means of sequencing.

Confirmation of Sec- and Tat-dependent secretion of FbFPs and YFPs (SEQ ID NO: 4) was performed by means of fluorescence microscopy analysis, as well as by means of immunologic detection of protein accumulation by Western Blot.

To be able to detect secretion of FbFPs into the periplasmatic space, the localization of the fluorescence marker was determined optically using the fluorescence microscope (Zeiss Axioplan 2 imaging with Apotome, lens Apochromat 100 oil 1.4; fluorescence filter Ex: 380/14 Em: 494/20) in vivo. For this purpose the constructs were grown overnight in E. coli strain MC4100, and in E. coli strain DADE in auto induction medium (5 g/l glycerol, 12 g/l tryptone, 24 g/l yeast extract, 2.32 g/l KH₂PO₄, 12.5 g/l K₂HPO₄ (pH 7.2), lactose 2 g/l, glucose 0.5 g/l) and the antibiotic chloramphenicol, which attains an automatic induction of directed gene expression, as soon as the glucose available has been metabolized, and the Lac promoter is no longer being inhibited, but rather induced by the lactose. To be able to determine that the secretion marker constructs, which are fused to the signal sequence of the Pelb, are being exclusively translocated via the Sec-secretion pathway and do not additionally reach the periplasm via the Tat secretion pathway, the DADE strain is a TatA-E deletion mutant.

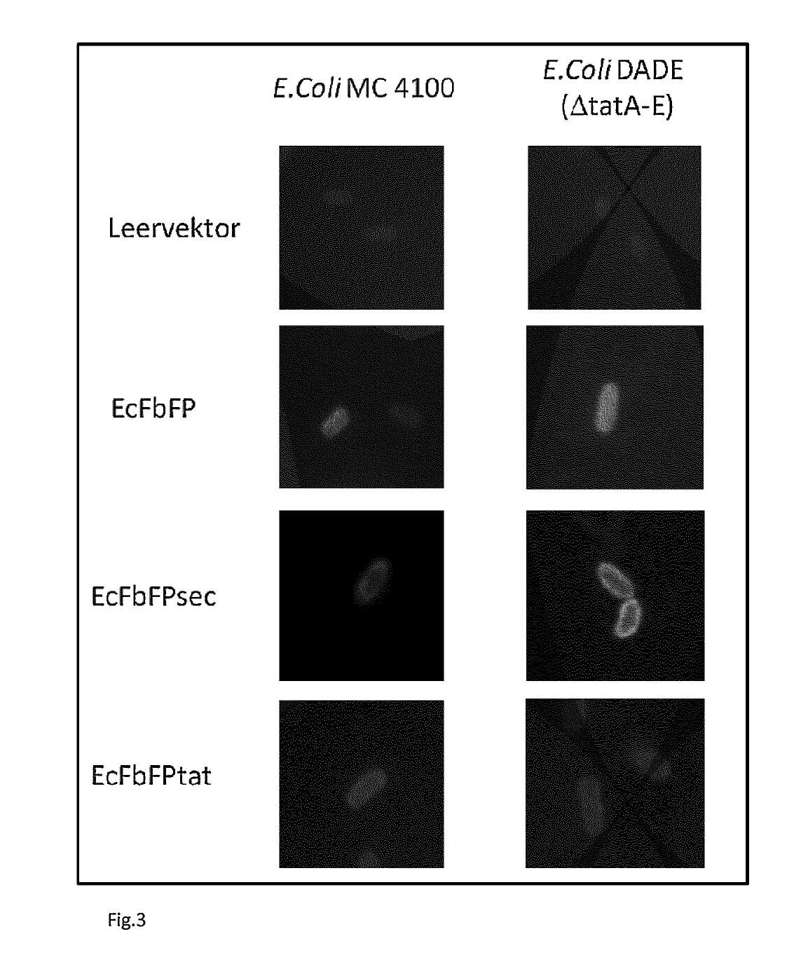

FIG. 3 shows a localisation analysis by means of a fluorescence microscope. Fluorescence recordings of the respective secretion constructs of EcFbFP (SEQ ID NO: 1), including the expression control (LOV), in E. coli MC in 4100 and in DADE cells are depicted. 3 μl of cell culture were examined in each case with an o.d.580=1.5. (100-fold magnification).

In FIG. 3 clear differences in the localisation of the EcFbFP (SEQ ID NO: 1) can be recognised when compared to the FbFP with a Sec- and a Tat-signal sequence, respectively. While the FbFPs without signal sequence are evenly distributed in the cytoplasm of the E. coli cell, the FbFPs with Sec- and Tat-signal sequence are only detectable in the outer ring of the respective cells. It can furthermore be recognised that the EcFbFPtat (SEQ ID NO: 3) proteins in the DADE mutant do not reach in the periplasm, whereas the secretion marker proteins in the wild type cells MC 4100 are very well being translocated. This points to a sec/Tat mediated localisation of the EcFbFPs (SEQ ID NO: 1) in the periplasm. It could thus be shown that the fluorescence markers of the FbFP family for the first time allow analysing secretory processes of the Sec- and Tat-pathway in bacteria.

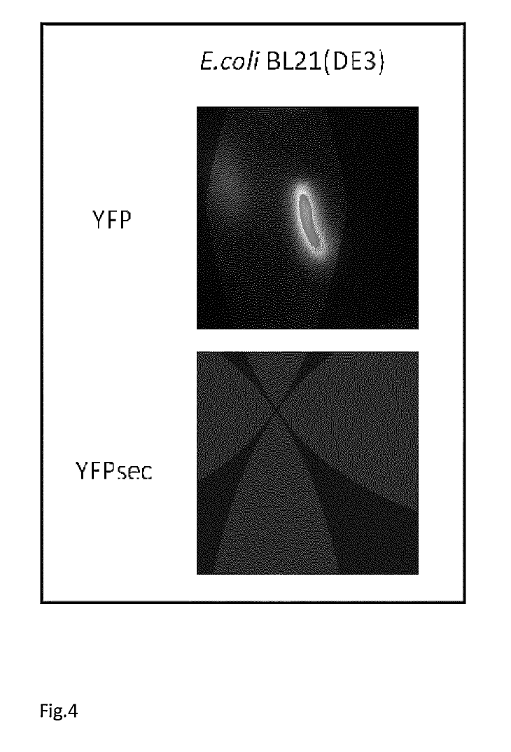

As a control, the YFP (SEQ ID NO: 4) and the YFP (SEQ ID NO: 5) were expressed in E. coli BL21 (DE3) cells in the expression vector pRhotHi-2 in auto induction medium, and potential fluorescences were detected by fluorescence microscope to be able to exclude secretion ability of the YFP (SEQ ID NO: 4) via the Sec-pathway. As can be taken from FIG. 4, only in the case of cytoplasmatic YFPs (SEQ ID NO: 4) in the expression control, expected active fluorescence occurred. As expected, in case of the YFP (SEQ ID NO: 5) no fluorescence is detectable, suggesting that YFP (SEQ ID NO: 4) is not suitable as a secretion marker as it does not exist in an active fluorescent form in the periplasm.

1. (canceled)

2. The method according to claim 12, wherein at least one cysteine in the LOV domain of the fluorescent protein is replaced by alanine.

3. The method according to claim 12, wherein the LOV domain of the fluorescent protein comprises at least one further point mutation in addition to the exchange of the at least one cysteine.

4. The method according to claim 12, wherein the fluorescent protein comprising the LOV domain that
(a) is encoded by a nucleic acid of SEQ ID NO: 1 or a fragment, a variant, a homolog, or a derivative of this sequence;
(b) is encoded by a nucleic acid capable of hybridizing to the nucleic acid of (a) under stringent conditions;
(c) is encoded by a nucleic acid of at least 70% identity, preferably 95% identity, to a nucleic acid of (a) or (b);
(d) is encoded by a nucleic acid capable of hybridizing to a nucleic acid complementary to one of the nucleic acids of (a)-(c) under stringent conditions;
(e) is encoded by a nucleic acid comprising at least one silent mutation of a single nucleotide (as permitted by the degeneracy of the genetic code) when compared to the nucleic acids of (a)-(d);
(f) is encoded by a nucleic acid, the code of which is optimized for a particular expression system when compared to the nucleic acids of (a)-(e).

5. The method according to claim 12, wherein the fluorescent protein comprises a size between ≧16 kDa and ≦19 kDA.

6. The method according to claim 12, wherein the fluorescent protein comprises an excitation wavelength between ≧430 nm and ≦470 nm.

7. The method according to claim 12, wherein the fluorescent protein comprises an emission maximum between ≧470 nm and ≦520 nm.

8. The method according to claim 12, wherein the fluorescent protein is being expressed or co-expressed in a host cell, and being secreted into the periplasm and/or into an extracellular media.

9. The method according to claim 12, wherein the fluorescent protein comprises a signal sequence at its N-terminus.

10. The method according to claim 9, wherein the signal sequence is a PelB or a TorA signal sequence.

11. The method according to claim 9, wherein the fluorescent protein comprising the LOV domain that
(g) is encoded by a nucleic acid of SEQ ID NO: 2 or 3, or a fragment, a variant, a homolog, or a derivative of one of these sequences;
(h) is encoded by a nucleic acid capable of hybridizing to the nucleic acid of (a) under stringent conditions;
(i) is encoded by a nucleic acid of at least 70% identity, preferably 95% identity, to a nucleic acid of (a) or (b);
(j) is encoded by a nucleic acid capable of hybridizing to a nucleic acid complementary to one of the nucleic acids of (a)-(c) under stringent conditions;
(k) is encoded by a nucleic acid comprising at least one silent mutation of a single nucleotide (as permitted by the degeneracy of the genetic code) when compared to the nucleic acids of (a)-(d);
(l) is encoded by a nucleic acid, the code of which is optimized for a particular expression system when compared to the nucleic acids of (a)-(e).

12. A method for labelling an antibody expressed in an organism, wherein the method comprises
labelling the antibody with a fluorescent protein comprising a LOV domain, in which at least one cysteine is replaced by another amino acid, which does not covalently bind to FMN and
detecting secretion or localization of the antibody into periplasm or extracellular space of the organism by means of excitation of the fluorescent protein by light.

13. The method according to claim 12, wherein the light has a wavelength between ≧430 nm and ≦470 nm.

14. The method according to claim 12, wherein the fluorescent protein is expressed in a bacteria selected from the group consisting of Escherichia coli, Rhodobacter capsulatus, Pseudomonas putida and Bacillus subtilis.

15. The method according to claim 12, wherein the fluorescent protein is expressed in a vector selected from the group consisting of pRhotHi-2 and pHSG575.

16. The method according to claim 6, wherein the excitation wavelength is 450 nm.

17. The method according to claim 7, wherein the emission maximum is 495 nm.

18. The method according to claim 13, wherein the light has a wavelength of 450 nm.

19. The method according to claim 14, wherein the fluorescent protein
(g) is encoded by a nucleic acid of SEQ ID NO: 1, 2 or 3, or a fragment, a variant, a homolog, or a derivative of one of these sequences;
(h) is encoded by a nucleic acid capable of hybridizing to the nucleic acid of (a) under stringent conditions;
(i) is encoded by a nucleic acid of at least 70% identity, preferably 95% identity, to a nucleic acid of (a) or (b);
(j) is encoded by a nucleic acid capable of hybridizing to a nucleic acid complementary to one of the nucleic acids of (a)-(c) under stringent conditions;
(k) is encoded by a nucleic acid comprising at least one silent mutation of a single nucleotide (as permitted by the degeneracy of the genetic code) when compared to the nucleic acids of (a)-(d);
(l) is encoded by a nucleic acid, the code of which is optimized for a particular expression system when compared to the nucleic acids of (a)-(e).
